(19) United States
(12) Patent Application Publication
(10) Pub. No.: US 2022/0096278 A1
(43) Pub. Date: Mar. 31, 2022

(54) Wound Dressing

(71) Applicant: Allgen Company, Taichung City (TW)

(72) Inventor: Chi-Chao Kuo, Taichung City (TW)

(21) Appl. No.: 17/230,022

(22) Filed: Apr. 14, 2021

(30) Foreign Application Priority Data
Sep. 28, 2020 (TW) 109133712

(51) Int. Cl. A61F 13/06; A61F 13/10

(52) U.S. Cl. CPC A61F 13/061; A61F 13/068; A61F 13/105; A61F 13/101

(57) ABSTRACT

The present invention discloses a wound dressing, which includes a carrier, a dressing layer and an adhesive layer. The carrier includes a central portion, a plurality of fixed portions, and a plurality of slashes. The central portion has a geometric center. The fixed portions are disposed around the central portion, and the number of the fixed portions is between 3 and 20. Each of the slashes is located between two fixed portions. The carrier has a first length, and each of the slashes has a second length. The first length is the shortest distance from an outer edge of a fixed portion to the geometric center. The ratio of the second length to the first length is between 0.2 and 0.8. The dressing layer is disposed on the central portion and includes a hydrogel. The adhesive layer is disposed on the fixed portions.

### BACKGROUND

### 1. Technical Field

The present invention relates to a wound dressing.

### 2. Description of the Related Art

Wounds (e.g., lacerations, abrasions, punctures and burns) are generally treated by allowing natural healing or by using gauze, cotton pads or other wrapping materials to cover the wounds. However, these treatments allow the wound to have contact with the air, so the surface of the wound can easily become dry, and dryness can increase the time required for the wound to heal and may lead to scarring. Therefore, many kinds of wound dressings which can keep a wound in a slightly humid environment are available on the market, some examples being film dressings, foam dressings, hydrophilic dressings, or antibacterial dressings, so consumers can choose different kinds of wound dressings according to the types of wounds.

The aforementioned different kinds of wound dressings all have flat and multi-layer structures. It is suitable to use the aforementioned wound dressings to cover wounds on flat and large areas, such as on the arms, legs, or torso. However, the aforementioned wound dressings do not allow easy coverage of wounds on joints, fingers, toes, or other locations that require frequent movement or are small in area. In addition to the problem of areas where the wound dressing cannot be adhered, such dressings are more likely to allow air to contact the wound, so the wound cannot be kept in a moist environment and the effect of the wound dressing on promoting wound healing is reduced. Therefore, the prior art still has room for improvement.

### SUMMARY

In view of the aforesaid drawbacks of the prior art, it is an objective of the present disclosure to provide a wound dressing which comprises a carrier having a plurality of fixed portions to overcome drawbacks of the prior art, namely, that the conventional wound dressing cannot easily be attached to an area that requires frequent activity or that is small.

In order to achieve the above objectives, the present disclosure provides a wound dressing, which is used to adhere to a non-planar wound. The wound dressing comprises a carrier, a dressing layer, an adhesive layer and a release paper layer. The carrier comprises a central portion, a plurality of fixed portions and a plurality of slashes. The central portion has a geometric center. The fixed portions are disposed around the central portion, and the number of the fixed portions is between 3 and 20. Each of the slashes is located between two fixed portions. The carrier has a first length, and each of the slashes has a second length. The first length is a shortest distance from an outer edge of a fixed portion to the geometric center, and the second length is a length of each of the slashes. The ratio of the second length to the first length is between 0.2 and 0.8. The dressing layer is disposed on the central portion and comprises a hydrogel. The adhesive layer is disposed on the fixed portions. The release paper layer is disposed on a surface of the dressing layer and the adhesive layer opposite to the carrier. With the above structures, after the release paper layer is removed from the dressing layer and the adhesive layer, the dressing layer can be applied to cover a non-planar wound such that one side of one of the fixed portions attaches to one side of the adjacent fixed portion through the adhesive layer and the carrier presents a three-dimensional structure.

In an embodiment of the present disclosure, the carrier further comprises a spacing portion located between the central portion and the fixed portion.

In an embodiment of the present disclosure, the width of the spacing portion is between 0.05 cm and 1.5 cm.

In an embodiment of the present disclosure, the carrier is a round shape, and the geometric center of the central portion is a center of the carrier.

In an embodiment of the present disclosure, the spacing portion is a ring shape.

In an embodiment of the present disclosure, the slashes extend linearly from the outer edge of the fixed portions toward the center.

In an embodiment of the present disclosure, the area of each of the fixed portions is substantially the same.

In an embodiment of the present disclosure, the number of the slashes is the same as the number of the fixed portions.

In an embodiment of the present disclosure, the carrier further comprises an elastic fiber layer, and the dressing layer and the adhesive layer are disposed above the elastic fiber layer.

In an embodiment of the present disclosure, the carrier further comprises a polymer material layer, and the elastic fiber layer is disposed above the polymer material layer.

In an embodiment of the present disclosure, the hydrogel comprises hydroxyethyl methacrylate, glycerol and polyethylene glycol.

As above, the wound dressing of the present invention comprises a carrier, a dressing layer, an adhesive layer and a release paper layer. The carrier has a central portion, a plurality of fixed portions and a plurality of slashes. The dressing layer is disposed on the central portion, and the adhesive layer is disposed on the fixed portions. The fixed portions can be overlapped with the adjacent fixed portion and the carrier presents a three-dimensional structure such that the wound dressing can be flatly attached to a non-planar wound (e.g., a wound on a joint, finger, toe, etc.). Further, the number limitation of the fixed portions (between 3 and 20) and the size limitation of the slashes (the ratio of the second length to the first length being between 0.2 and 0.8) ensure that the area of the adhesive layer of each of the fixed portions is sufficient to adhere to the skin and to the adjacent fixed portion firmly for easy dressing.

In addition, the dressing layer has the hydrogel to keep the wound (i.e., the non-planar wound) in a moist environment that is conducive to wound healing and effectively relieve trauma wounds and burns.

### BRIEF DESCRIPTION OF THE DRAWINGS

In the drawings, wherein similar reference numerals denote similar elements throughout the several views:

FIG. 1 illustrates a perspective view of a wound dressing according to an embodiment of the present invention;

FIG. 2 illustrates a top view of the carrier shown in FIG. 1;

FIG. 3 illustrates an exploded view of the wound dressing shown in FIG. 1;

FIG. 4 illustrates a schematic view of the wound dressing shown in FIG. 1 adhered to a non-planar wound; and

FIG. 5 illustrates a top view of a wound dressing according to another embodiment of the present invention.

### DETAILED DESCRIPTION OF THE EMBODIMENTS

In order to make the structure and characteristics as well as the effectiveness of the present disclosure further understood and recognized, a detailed description of the present disclosure is provided as follows along with embodiments and accompanying figures.

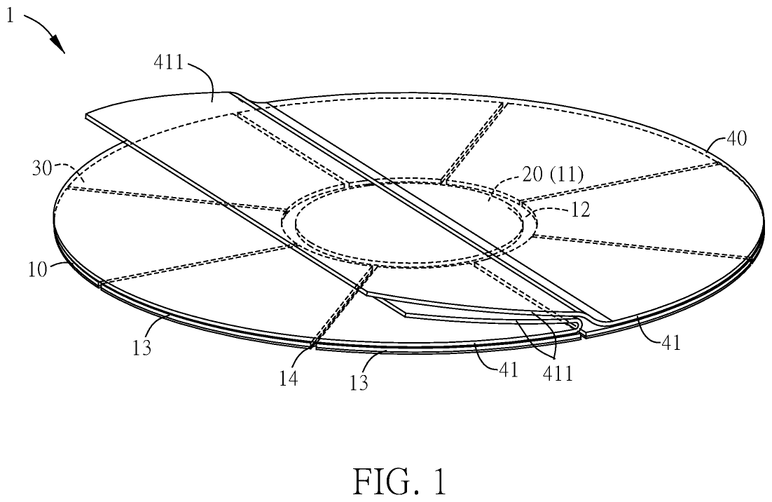

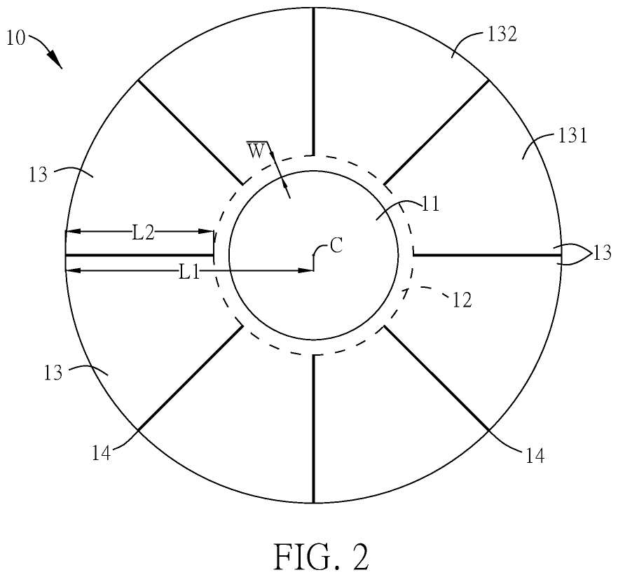

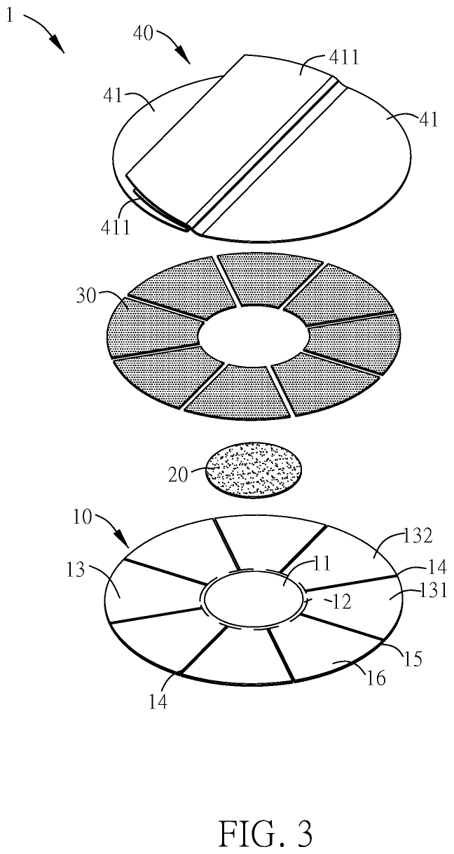

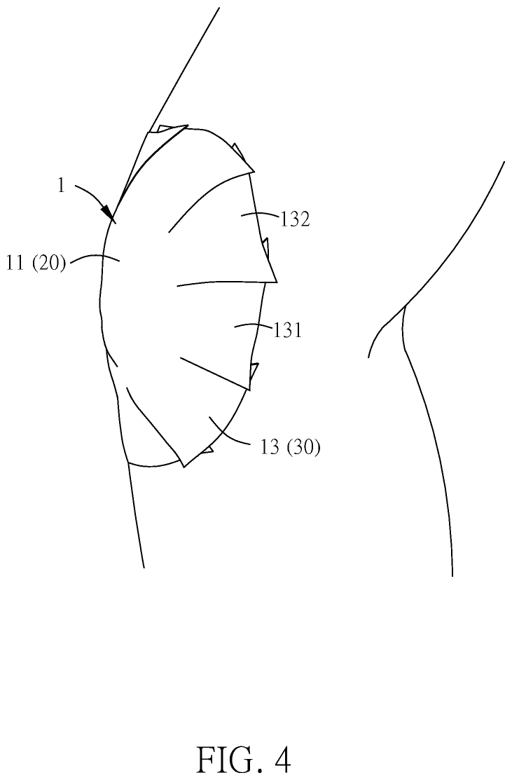

FIG. 1 illustrates a perspective view of a wound dressing according to an embodiment of the present invention, FIG. 2 illustrates a top view of the carrier shown in FIG. 1, and FIG. 3 illustrates an exploded view of the wound dressing shown in FIG. 1. Please refer to FIG. 1, FIG. 2 and FIG. 3. First, the wound dressing 1 of this embodiment is designed to adhere to a non-planar wound. Further, in this embodiment, the non-planar wound is a wound on the surface of a joint, such as the elbow, heel, or knee (as shown in FIG. 4), or on fingers or toes, which are small in area.

Please refer to FIG. 1 and FIG. 3. In this embodiment, the wound dressing 1 comprises a carrier 10, a dressing layer 20, an adhesive layer 30 and a release paper layer 40. The dressing layer 20 and the adhesive layer 30 are disposed on a surface of the carrier 10, and the release paper layer 40 is disposed on the surfaces of the dressing layer 20 and the adhesive layer 30 opposite to the carrier 10. As shown in FIG. 3, the top layer of the wound dressing 1 is the release paper layer 40, the middle layer of which is the dressing layer 20 and the adhesive layer 30 located in the same layer, and the bottom layer of which is the carrier 10.

Referring to FIG. 2, the carrier 10 comprises a central portion 11, a spacing portion 12, a plurality of fixed portions 13 and a plurality of slashes 14. The central portion 11 is located at a central area of the carrier 10 and has a geometric center C. In this embodiment, for example, the central portion 11 is a round shape, so the geometric center C is the center of the central portion 11. Preferably, the carrier 10 of this embodiment is also a round shape such that the geometric center C of the central portion 11 is also the center of the carrier 10. That is, the carrier 10 and the central portion 11 have the same geometric center C (center). The spacing portion 12 is located at an outer edge of the central portion 11, and the fixed portions 13 extend outward from the spacing portion 12. In other words, the fixed portions 13 are disposed around the central portion 11, and the spacing portion 12 is located between the central portion 11 and the fixed portions 13.

The two adjacent fixed portions 13 are not connected to each other but are connected to the spacing portion 12 by one end of the fixed portions 13. Specifically, in this embodiment, the fixed portions 13 and the slashes 14 are formed by cutting the carrier 10. Each of the slashes 14 is located between two adjacent fixed portions 13, and the number of the slashes 14 is the same as that of the fixed portions 13. It should be noted that the central portion 11, the spacing portion 12 and the fixed portions 13 of the carrier 10 are virtual areas for facilitating the cutting and coating of the dressing layer 20 and the adhesive layer 30.

Since the slashes 14 are located between the fixed portions 13, one of the fixed portions 13 (e.g., the fixed portion 131) is able to overlap with the adjacent fixed portion 13 (e.g., the fixed portion 132) such that the carrier 10 presents a three-dimensional structure and the wound dressing 1 can be applied to a non-planar wound. Specifically, the dressing layer 20 is disposed on the central portion 11, and the adhesive layer 30 is disposed on the fixed portions 13. In other words, the dressing layer 20 is coated on the central portion 11 of the carrier 10, and the adhesive layer 30 is coated on the fixed portions 13. In another embodiment, the adhesive layer 30 may be coated on the entire surface of the carrier 10. For example, after the dressing layer 20 is coated on the central portion 11, the adhesive layer 30 is directly coated on a surface which has the dressing layer 20 of the carrier 10, such that the outside surfaces of the fixed portions 13, the spacing portion 12 and the dressing layer 20 are all coated with the adhesive layer 30.

The release paper layer 40 is disposed on the surfaces of the dressing layer 20 and the adhesive layer 30 opposite to the carrier 10. When the wound dressing 1 is not used, the release paper layer 40 is able to prevent the dressing layer 20 from being contaminated and prevent the adhesive layer 30 from adhering to other areas. The release paper layer 40 includes a single or multiple (preferably two) release papers 41. A smooth coating layer (usually an organic silicone resin type release agent) of the release paper 41 adheres to the adhesive layer 30. Further, the area of the release paper layer 40 is greater than or equal to that of the carrier 10. In this embodiment, the release paper layer 40 has two release papers 41, and one of the release papers 41 has a bending portion 411. The bending portion 411 is folded back from the release paper 41 to the surface opposite to the adhesive layer 30 such that bending portion 411 does not adhere to the adhesive layer 30 and the user can easily remove the release paper layer 40. The other one of the release papers 41 also has a bending portion 411, which is overlapped above the folded bending portion 411.

Please refer to FIG. 4. FIG. 4 illustrates a schematic view of the wound dressing shown in FIG. 1 adhered to a non-planar wound. When using the wound dressing 1, the user can pinch the bending portion 411 and then remove the release paper layer 40 from the adhesive layer 30. That is, the release paper layer 40 is separated from the dressing layer 20 and the adhesive layer 30. Then the dressing layer 20 (central portion 11) corresponds to and covers the non-planar wound (such as a wound on the elbow). After that, one of the fixed portions 13 (e.g., the fixed portion 131) is adhered to the skin near the non-planar wound, and then one side of the adjacent fixed portion 13 (e.g., the fixed portion 132) is adhered to one side of the aforesaid fixed portions 13 (i.e., the fixed portion 131) by the adhesive layer 30. The operations are repeated to adhere all the fixed portions 13 so that one side of each of the fixed portions 13 can be adhered to one side of the adjacent fixed portion 13. At this time, the carrier 10 presents a three-dimensional structure, as shown in FIG. 4, for flatly adhering to the elbow.

In this embodiment, the dressing layer 20 comprises a hydrogel which can keep the wound (a non-planar wound) in a moist environment to facilitate wound healing. The hydrogel comprises moisturizing ingredients, which may be, but are not limited to, hydroxyethyl methacrylate (2-HEMA), glycerol and polyethylene glycol (PEG). In addition, the hydrogel further comprises pure water and polyvinylpyrrolidones which can form a colloidal structure (or act as a thickening agent). Moreover, the hydrogel can effectively relieve trauma wounds and burns. The signals released by nerve cells in a moist environment are weaker than those released by nerve cells in a dry environment, so the hydrogel can effectively reduce the pain caused by the nerve cells during the wound healing.

Because the spacing portion 12 is disposed on the outside of the central. portion 11, then even if the hydrogel leaks slightly, it will be contained by the spacing portion 12. In other words, the spacing portion 12 can prevent the hydrogel from leaking out of the wound dressing 1 from the non-connected portions (i.e., the slashes 14) between the fixed portions 13. Preferably, the width W of the spacing portion 12 may be between 0.05 cm and 1.5 cm, and more preferably between 0.2 cm and 1 cm, such as 0.5 cm. The width limitation can prevent the leakage of the hydrogel of the dressing layer 20 effectively.

In addition, the adhesive layer 30 may be a pressure sensitive adhesive (PSA), which is easy for the user to adhere and tear off. Preferably, the PSA may be made of low sensitivity materials.

In this embodiment, the number and the size of the fixed portions 13 are limited to a specific range such that the carrier 10 can form a three-dimensional structure, as shown in FIG. 3. In this embodiment, the number of the fixed portions 13 is between 3 and 20; that is, the carrier 10 has 3 to 20 fixed portions 13. The lower limit of the number of the fixed portions 13 (i.e., 3) ensures that the carrier 10 can form a three-dimensional structure by overlapping of the fixed portions 13 with each other, and the three-dimensional structure can cover and be attached to the non-planar wound. Preferably, the number of the fixed portions 13 is between 3 and 8. The upper limit of the fixed portions 13 (i.e., 8) can avoid excessive operation (adhering) steps.

As shown in FIG. 2, the carrier 10 has a first length L1, and each of the slashes 14 has a second length L2. The first length L1 is the shortest distance from an outer edge of each of the fixed portions 13 to the geometric center, and the second length is the length of each of the slashes 14. In this embodiment, the ratio of the second length L2 to the first length L1 is between 0.2 and 0.8, preferably between 0.25 and 7, and more preferably between 0.4 and 0.6. Specifically, the carrier 10 of this embodiment has a round shape, so the first length L1 is a radius of the carrier 10. Preferably, the central portion 11, the spacing portion 12 and the fixed portions 13 are arranged in a concentric circle as a whole, so the spacing portion 12 presents a ring shape. Further, the slashes 14 extend linearly from the outer edge of the fixed portions 13 toward the geometric center C (i.e., the center) such that each of the fixed portions 13 presents a sector shape or a sector-like shape.

The second length L2 is the length of each of the slashes 14. When designing the wound dressing 1, the designer can define the first length L1 according to a proper area of each of the fixed portions 13 and then estimate a numerical range of the second length L2 according the limitation that the ratio of the second length L2 to the first length L1 is between 0.2 and 0.8. For example, if the design requirement is that the diameter of the carrier 10 (wound dressing 1) is 12 cm, the first length L1 is 6 cm and the second length L2 is between 1.2 cm and 4.8 cm, preferably between 1.5 cm and 4.2 cm, and more preferably between 2.4 cm and 3.6 cm. The length limitation and the aforementioned number limitation of the fixed portions 13 and the slashes 14 ensure that the area of the adhesive layer 30 on each of the fixed portions 13 is sufficient to stably adhere to the skin and the adjacent fixed portion 13. Preferably, the areas of each of the fixed portions 13 are substantially the same.

Preferably, as shown in FIG. 3, the carrier 10 comprises a polymer material layer 15 and an elastic fiber layer 16. The dressing layer 20 and the adhesive layer 30 are disposed above the elastic fiber layer 16. As shown in FIG. 3, the polymer material layer 15 is at the bottom, the elastic fiber layer 16 is disposed above the polymer material layer 15, and the dressing layer 20 and the adhesive layer 30 are coated on the elastic fiber layer 16. The polymer material layer 15 may be, but is not limited to, a polyurethane (PU) layer. The polymer material layer 15 is waterproof, breathable and leakproof, and the elastic fiber layer 16 has the characteristic of unidirectional extension.

After the dressing layer 20 located on the central portion 11 covers the non-planar wound, the elasticity of the polymer material layer 15 and the elastic fiber layer 16 allows the the user to pull one of the fixed portions 13 outward and then adhere it to the skin with the adhesive layer 30. In addition, after the hydrogel of the adhesive layer 30 absorbs the liquid (e.g., the tissue fluid from the wound) and swells, the polymer material layer 15 and the elastic fiber layer 16 can provide downward pressure (toward the wound) by the elasticity thereof, so the wound will heal flatly and not easily leave a scar.

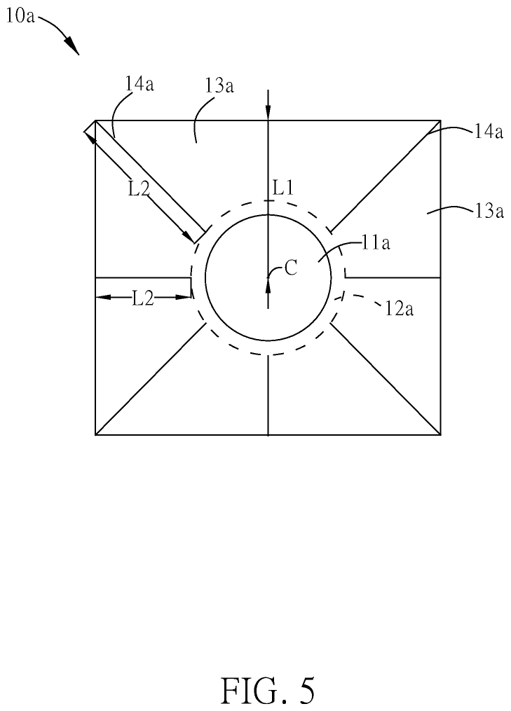

Please refer to FIG. 5. FIG. 5 illustrates a top view of a wound dressing 10a according to another embodiment of the present invention. The present invention does not limit the shape of the carrier 10a, which may be other polygons, such as a quadrilateral shape. The central portion 11a is also a round shape and has a geometric center C. The present embodiment does not limit the shape of the spacing portion 12a, which may be a ring shape as in the aforementioned embodiment or an irregular shape with an inner circle and outer square. Each of the fixed portions 13a is similar to a triangle shape without one corner (the missing corner close to the central portion 11a and the spacing portion 12a). The number of the fixed portions 13a is between 3 and 20 and preferably between 3 and 8. The first length L1 is a vertical length from the geometric center C to the outer edge of one of the fixed portions 13a, and the second length L2 is the length of the slashes 14a. Similarly, the ratio of the second length L2 to the first length L1 is between 0.2 and 0.8.

As above, the wound dressing of the present invention comprises a carrier, a dressing layer, an adhesive layer and a release paper layer. The carrier has a central portion, a plurality of fixed portions and a plurality of slashes. The dressing layer is disposed on the central portion, and the adhesive layer is disposed on the fixed portions. The fixed portions can be overlapped with the adjacent fixed portions such that the carrier presents a three-dimensional structure and the wound dressing can be flatly attached to a non-planar wound (e.g., a wound on a joint, finger, or toe). Further, the number limitation of the fixed portions (between 3 and 20) and the size limitation of the slashes (the ratio of the second length to the first length being between 0.2 and 0.8) ensure that the area of the adhesive layer of each of the fixed portions is sufficient to adhere to the skin and to the adjacent fixed portion firmly for easy dressing.

In addition, the dressing layer has the hydrogel to keep the wound (i.e., the non-planar wound) in a moist environment that is conducive to wound healing and effectively relieve trauma wounds and burns.

It should be noted that the described embodiments are only for illustrative and exemplary purposes and that various changes and modifications may be made to the described embodiments without departing from the scope of the application as disposed by the appended claims.

What is claimed is:

1. A wound dressing, used to adhere to a non-planar wound, the wound dressing comprising:
a carrier comprising:
a central portion having a geometric center;
a plurality of fixed portions disposed around the central portion, the number of the fixed portions being between 3 and 20; and
a plurality of slashes, each of the slashes being located between the fixed portions,
wherein the carrier has a first length, each of the slashes has a second length, the first length is the shortest distance from an outer edge of each of the fixed portions to the geometric center, the second length is the length of each of the slashes, and the ratio of the second length to the first length is between 0.2 and 0.8;
a dressing layer disposed on the central portion and comprising a hydrogel; and
an adhesive layer disposed on the fixed portions; and
a release paper layer disposed on a surface of the dressing layer and the adhesive layer opposite to the carrier,
wherein after the release paper layer is removed from the dressing layer and the adhesive layer, the dressing layer covers the non-planar wound and one side of one of the fixed portions attaches to one side of the adjacent fixed portion through the adhesive layer such that the carrier presents a three-dimensional structure.

2. The wound dressing as claimed in claim 1, wherein the carrier further comprises a spacing portion located between the central portion and the fixed portion.

3. The wound dressing as claimed in claim 2, wherein the width of the spacing portion is between 0.05 cm and 1.5 cm.

4. The wound dressing as claimed in claim 1, wherein the carrier is a round shape, and the geometric center of the central portion is the center of the carrier.

5. The wound dressing as claimed in claim 4, wherein the spacing portion is a ring shape.

6. The wound dressing as claimed in claim 5, wherein the slashes extend linearly from the outer edge of the fixed portions toward the center.

7. The wound dressing as claimed in claim 5, wherein the area of each of the fixed portions is substantially the same.

8. The wound dressing as claimed in claim 1, wherein the number of the slashes is the same as the number of the fixed portions.

9. The wound dressing as claimed in claim 1, wherein the carrier further comprises an elastic fiber layer, and the dressing layer and the adhesive layer are disposed above the elastic fiber layer.

10. The wound dressing as claimed in claim 9, wherein the carrier further comprises a polymer material layer, and the elastic fiber layer is disposed above the polymer material layer.

11. The wound dressing as claimed in claim 1, wherein the hydrogel comprises hydroxyethyl methacrylate, glycerol and polyethylene glycol.
